(19) United States
(12) Patent Application Publication
(10) Pub. No.: US 2014/0112971 A1
(43) Pub. Date: Apr. 24, 2014

(54) Edible Product

(71) Applicants: Amita Shukla, Ellicott City, MD (US); Mukta Shukla, Ellicott City, MD (US)

(72) Inventors: Amita Shukla, Ellicott City, MD (US); Mukta Shukla, Ellicott City, MD (US)

(21) Appl. No.: 13/657,851

(22) Filed: Oct. 22, 2012

(51) Int. Cl. A23L 1/30; A61K 36/9068; A61K 36/9066

(52) U.S. Cl. CPC A23L 1/3002; A61K 36/9066; A61K 36/9068
USPC 424/439; 424/756

(57) ABSTRACT

The present invention describes an orally administered edible product designed to provide a health benefit in a delivery formulation that will increase ease of use and thus lead to increased compliance and consumption to promote human health and wellness.

The present invention provides an enhanced oral delivery method by developing edible products that can be ingested like food, are designed to taste like food not medicine, provide optimal absorption of lipophilic ingredients and allow for the delivery of the equivalent of multiple capsules in a single oral “dose” thus providing a superior method for the consumption of plant-based products.

### BACKGROUND OF THE INVENTION

Most health and wellness products made for oral consumption are currently available in pill, capsule or liquid delivery formats. These include products such as vitamins, minerals, nutritional supplements, nutraceuticals and plant-derived products such as herbs and spices. These oral products are consumed by humans because they are believed to confer health benefits through the ingested ingredients.

But, there are several shortcomings to the current delivery methods for these health and wellness products. Pills and capsules can be difficult to swallow and increase the pill burden for individuals who may already be consuming other orally administered pills such as medicines. Most people do not like to consume pills, especially if multiple pills or capsules have to be consumed at the same time.

Also, many liquid formulations of health products such as vitamin-containing drinks, syrups and energy shots either require the consumption of significant volumes of fluid and/or they do not have a desirable taste. This reduces many people's desire to consume and comply with regularly taking health and wellness products that may confer health benefits if consumed as intended.

In addition, for some plant derived products such as herbs and spices, the delivery in a capsule format may not optimize absorption of lipophilic active ingredients since such pills and capsules are typically consumed with water, leading to lower bioavailability of the active ingredients.

The present invention describes an orally administered edible product designed to provide a health benefit in a delivery formulation that will increase ease of use and thus lead to increased compliance and consumption to promote human health and wellness.

The present invention provides an enhanced oral delivery method by developing edible products that can be ingested like food, are designed to taste like food not medicine, provide optimal absorption of lipophilic ingredients and allow for the delivery of the equivalent of multiple capsules in a single oral “dose” thus providing a superior method for the consumption of plant-based products.

### BRIEF SUMMARY OF THE INVENTION

The present invention describes an orally administered edible product designed to provide a health benefit in a delivery formulation that will increase ease of use and thus lead to increased compliance and consumption to promote human health and wellness.

The present invention provides an enhanced oral delivery method by developing edible products that can be ingested like food, are designed to taste like food not medicine, provide optimal absorption of lipophilic ingredients and allow for the delivery of the equivalent of multiple capsules in a single oral “dose” thus providing a superior method for the consumption of plant-based products.

### BRIEF DESCRIPTION OF THE SEVERAL VIEWS OF THE DRAWING

The foregoing and still other objects of this invention will become apparent, along with various advantages and features of novelty residing in the present embodiments, from study of the following drawings, in which:

FIG. 1A is a sphere shaped embodiment of the edible product, according to the present invention in which said coating fully covers the surface of said mixture and FIG. 1B is a cross sectional view of FIG. 1A.

FIG. 2A is a oblate spheroid shaped embodiment of the edible product, according to the present invention in which said coating fully covers the surface of said mixture and FIG. 2B is a cross sectional view of FIG. 2A.

FIG. 3A is a cube shaped embodiment of the edible product, according to the present invention in which said coating fully covers the surface of said mixture and FIG. 3B is a cross sectional view of FIG. 3A.

FIG. 4A is a cube shaped embodiment of the edible product, according to the present invention in which said coating partially covers the surface of said mixture and FIG. 4B is a cross sectional view of FIG. 4A.

### DETAILED DESCRIPTION OF THE INVENTION

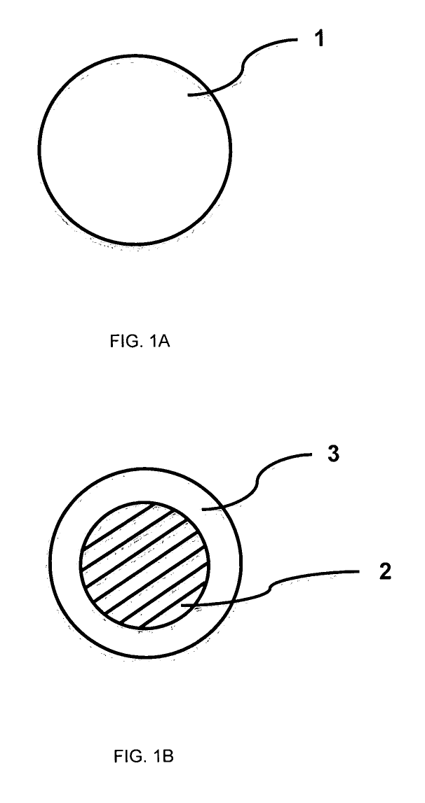

Referring to the drawings, FIG. 1A shows said edible product (1) in a spherical shape and FIG. 1B represents a cross-section of the embodiment of said edible product (1) shown in FIG. 1A. FIG. 1B also shows an embodiment of the mixture (2) according to the present invention with its surface fully covered by the coating (3), according to the present invention.

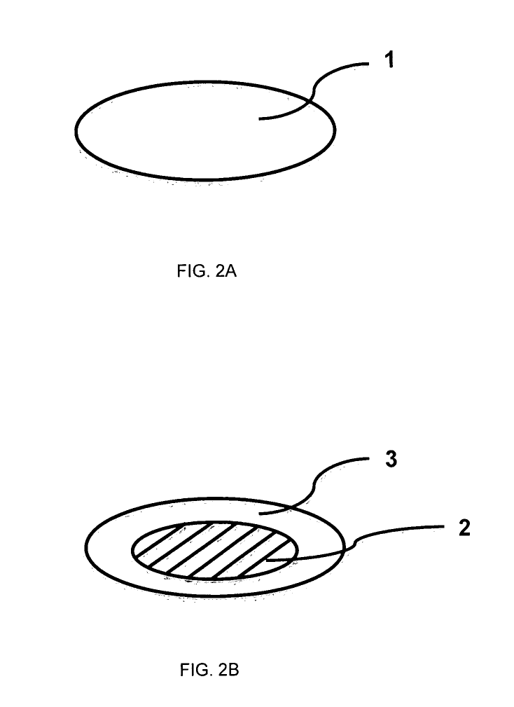

FIG. 2A shows said edible product (1) in a oblate spheroid shape and FIG. 2B represents a cross-section of the embodiment of said edible product (1) shown in FIG. 2A. FIG. 2B also shows an embodiment of the mixture (2) according to the present invention with its surface fully covered by the coating (3), according to the present invention.

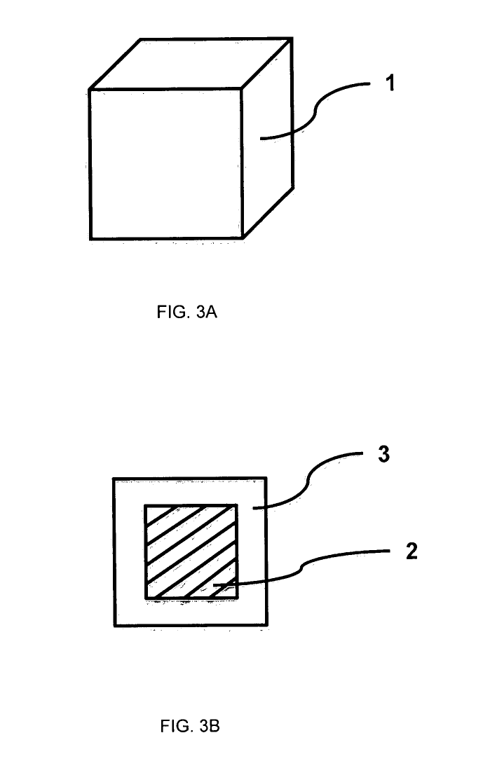

FIG. 3A shows said edible product (1) in a cube shape and FIG. 3B represents a cross-section of the embodiment of said edible product (1) shown in FIG. 3A. FIG. 3B also shows an embodiment of the mixture (2) according to the present invention with its surface fully covered by the coating (3), according to the present invention.

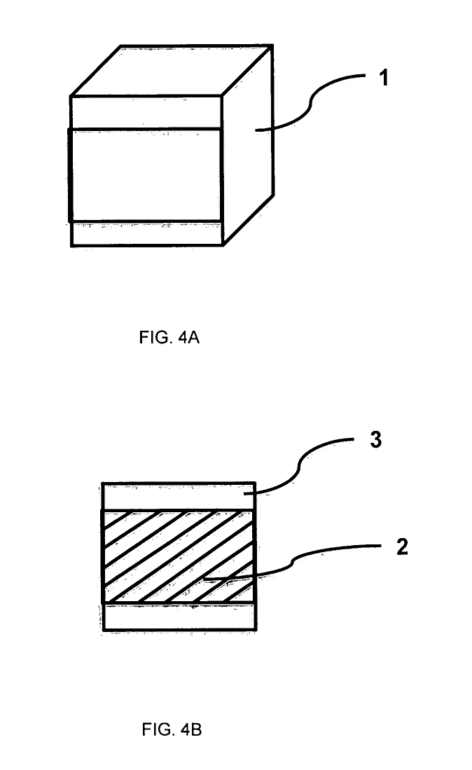

FIG. 4A shows said edible product (1) in a oblate spheroid shape and FIG. 4B represents a cross-section of the embodiment of said edible product (1) shown in FIG. 4A. FIG. 4B also shows an embodiment of the mixture (2) according to the present invention with its surface partially covered by the coating (3), according to the present invention.

As shown in the drawings, said edible product (1) can be of a shape selected from the group comprised of but not limited to: sphere, cube, rectangular cube, oblate spheroid, donut, pyramid, cone, disc, cylinder and combinations thereof.

The edible product (1) described in the present invention is designed to be ingested orally and is intended to have a therapeutic, curative or preventative health benefit. Said health benefit includes prevention or treatment of at least one disease or physical disorder selected from the group comprised of, but not limited to: inflammatory diseases, cardiovascular diseases, metabolic diseases, autoimmune diseases, bone and joint diseases, central nervous system diseases and endocrine disorders. Such diseases include but are not limited to depression, Alzheimer's disease, dementia, pain, blood pressure, inflammation, arthritis, rheumatoid arthritis, diabetes and obesity.

Said mixture (2) in the edible product (1) is comprised of at least one plant-based product and at least one lipid-based edible substance. Said mixture (2) can also contain at least one sweetener.

The plant-based product in said mixture (2) can be a crude preparation, paste, powder or extract derived selected from the group comprised of, but not limited to: the leaves, roots, stem, seeds, fruits, flowers, bark, nuts, tubers, tumors, other potentially edible components of a plant or tree, and mixtures thereof. The extract, as described herein can be comprised of selected active ingredients of said plant-based product.

The plant-based product in said mixture (2) is selected from the group comprised of, but not limited to, plant-derived products described in: Western herbal preparations, naturopathic medicine, Ayurveda, Unani medicine, Siddhi medicine, other systems of alternative plant based medicine, and any spices and herbs used in the preparation, preservation or cooking of edible foods, and mixtures thereof.

For example, said plant-based product can be a crude preparation, paste, powder or extract selected from the group comprised of, but not limited to: turmeric, ginger, cinnamon, black pepper, anise, fenugreek, cardamom, tulsi (Indian basil), cocoa, amla (Indian gooseberry), bhibitaki, haritaki, ashwagandha, bitter gourd, coriander, ajwain and mixtures thereof.

The lipid-based edible substance in said mixture (2) is selected from the group consisting of, but not limited to: ghee (clarified butter), butter, cocoa butter, almonds, almond butter, almond oil, peanuts, peanut butter, peanut oil, cashews, cashew butter, cashew oil, walnuts, walnut butter, walnut oil, sesame, sesame paste, sesame oil, sunflower seeds, sunflower paste, sunflower oil, pecans, pecan butter, pecan oil, coconuts, coconut oil, any plant or animal derived fat, and mixtures thereof.

Said mixture (2) may also include at least one sweetener, wherein said sweetener is selected from the group consisting of, but not limited to: honey, maple syrup, sugar, cane sugar, cane juice, beet sugar, brown sugar, stevia, palm sugar, coconut sugar, turbinado sugar, caramel, jaggery, molasses, dates, date syrup, raisins, fruit derived sweeteners, fructose and mixtures thereof.

Said mixture (2) can also contain at least additional substance selected from the group comprised of, but not limited to: vitamins, supplements, nutraceuticals, functional foods, GRAS food additives and combinations thereof.

Said mixture (2) can be an emulsion or any type of crude or blended mixture that permits for mixing of the plant-based product, lipid-based edible substance and other potential ingredients of said mixture.

Said mixture (2) is fully or partially enclosed in an edible coating (3). As shown in FIGS. 3b and 4b, said coating (3) may cover the surface of said mixture either fully or partially, respectively.

Said edible coating (3) is comprised of edible substances selected from the group comprised of, but not limited to: chocolate, milk chocolate, white chocolate, dark chocolate, cocoa-derived products, sugar, corn syrup, wax, candy, preservatives, cocoa powder, flour, rice, stabilizing agents, other grain based substances, gram flour and mixtures thereof.

### EXAMPLE

In one embodiment of the invention, a plant based mixture containing turmeric and ginger is mixed with a lipid-based substance comprised of almond butter and a sweetener comprised of sugar. The mixture is formed into a spherical shape and fully coated with chocolate.

While a specific embodiment of the invention has been shown and described in detail to illustrate the application of the principles of the invention, it is understood that the invention may be embodied otherwise without departing from such principles and that various modifications, alternate constructions, and equivalents will occur to those skilled in the area given the benefit of this disclosure and the embodiment described herein, as defined by the appended claims.

What is claimed is:

1. An edible product comprised of a mixture containing: (1) at least one plant-based product and (2) at least one lipid-based edible substance wherein said mixture is fully or partially enclosed in an edible coating.

2. The edible product of claim 1, wherein said edible product is intended to have a therapeutic, curative or preventative health benefit.

3. The health benefit of claim 2, wherein said health benefit includes prevention or treatment of at least one disease or physical disorder selected from the group comprised of, but not limited to: inflammatory diseases, cardiovascular diseases, metabolic diseases, autoimmune diseases, bone and joint diseases, central nervous system diseases and endocrine disorders.

4. The plant-based product of claim 1, wherein said plant based product can be a crude preparation, paste, powder or extract selected from the group comprised of, but not limited to: the leaves, roots, stem, seeds, fruits, flowers, bark, nuts, tubers, tumors, other potentially edible components of a plant or tree, and mixtures thereof.

5. The extract of claim 4 wherein said extract is comprised of selected active ingredients of said plant-based product.

6. The plant-based product of claim 1, wherein said plant-based product is selected from the group comprised of, but not limited to, plant-derived products described in: Western herbal preparations, naturopathic medicine, Ayurveda, Unani medicine, Siddhi medicine, other systems of alternative plant based medicine, and any spices and herbs used in the preparation, preservation or cooking of edible foods, and mixtures thereof.

7. The plant-based product of claim 1, wherein said plant-based product is a crude preparation, paste, powder or extract selected from the group comprised of, but not limited to: turmeric, ginger, cinnamon, black pepper, anise, fenugreek, cardamom, tulsi (Indian basil), cocoa, amla (Indian gooseberry), bhibitaki, haritaki, ashwagandha, bitter gourd, coriander, ajwain and mixtures thereof.

8. The lipid based edible substance of claim 1, wherein said substance is selected from the group consisting of, but not limited to: ghee (clarified butter), butter, cocoa butter, almonds, almond butter, almond oil, peanuts, peanut butter, peanut oil, cashews, cashew butter, cashew oil, walnuts, walnut butter, walnut oil, sesame, sesame paste, sesame oil, sunflower seeds, sunflower paste, sunflower oil, pecans, pecan butter, pecan oil, coconuts, coconut oil, any plant or animal derived fat, and mixtures thereof.

9. The edible product of claim 1, wherein said edible product contains at least one sweetener.

10. The sweetener of claim 9, wherein said sweetener is selected from the group consisting of, but not limited to: honey, maple syrup, sugar, cane sugar, cane juice, beet sugar, brown sugar, stevia, palm sugar, coconut sugar, turbinado sugar, caramel, jaggery, molasses, dates, date syrup, raisins, fruit derived sweeteners, fructose and mixtures thereof.

11. The edible product of claim 1, wherein said edible of product can be of a shape selected from the group comprised of but not limited to: sphere, cube, rectangular cube, oblate spheroid, donut, pyramid, cone, disc, cylinder and combinations thereof.

12. Said mixture of claim 1, wherein said mixture contains at least additional substance selected from the group comprised of, but not limited to: vitamins, supplements, nutraceuticals, functional foods, GRAS food additives and combinations thereof.

13. Said mixture of claim 1, wherein said mixture is an emulsion.

14. Said coating of claim 1, wherein said coating is comprised of edible substances selected from the group comprised of, but not limited to: chocolate, milk chocolate, white chocolate, dark chocolate, cocoa-derived products, sugar, corn syrup, wax, candy, preservatives, cocoa powder, flour, rice, stabilizing agents, other grain based substances, gram flour and mixtures thereof.

15. Said coating of claim 1, wherein said coating fully or partially covers the surface of said mixture.

16. The diseases of claim 3, wherein said disease or physical disorder is depression.
